(19) United States
(12) Patent Application Publication
(10) Pub. No.: US 2022/0346660 A1
(43) Pub. Date: Nov. 3, 2022

(54) SADDLE MRI

(71) Applicant: Weinberg Medical Physics, Inc., North Bethesda, MD (US)

(72) Inventor: Irving N. WEINBERG, Chevy Chase, MD (US)

(21) Appl. No.: 17/733,151

(22) Filed: Apr. 29, 2022

Related U.S. Application Data
(60) Provisional application No. 63/181,766, filed on Apr. 29, 2021.

(51) Int. Cl. A61B 5/055; G01R 33/383; A61B 5/00

(52) U.S. Cl. CPC A61B 5/055; G01R 33/383; A61B 5/70

(57) ABSTRACT

An apparatus and method are provided for pelvic imaging using a saddle shaped magnetic resonance imaging system. The apparatus includes an array of electropermanent magnets having a longitudinal length that is longer than a width so that the subject may straddle the length of the array and sit on it

### CROSS REFERENCE AND PRIORITY CLAIM

This application claims priority to U.S. Provisional Patent Application Ser. No. 63/181,766, entitled “SADDLE MRI,” filed 29 Apr. 2021, the entirety of which is incorporated by reference.

### FIELD

Disclosed embodiments are directed, generally, to an apparatus and method for pelvic imaging, in particular a saddle for seated magnetic resonance imaging.

The incidence of prostate cancer in African-American men is almost 60% higher than in white men, and the mortality rate is two- to three-times greater. Poor access to standardized healthcare has been shown to be an important contributor to this racial disparity. Behavioral research has shown that African-American men tend to see doctors less often than white men do, because of distrust, decreased economic resources, or other factors. African-American men are twice less likely to see a physician than white men.

The sensitivity and specificity of Prostate-Specific Antigen (PSA) for prostate cancer (with a typical threshold of 4 ng/mL applied) has been quoted at about 20% and 94% respectively, resulting in a 38% cancer miss rate. As of May 18, 2018, the US Preventive Services Taskforce (USPSTF) was equivocal in recommending screening for men 55-69 years of age (regardless of race) and recommended against screening of men 70 years or older. These recommendations were made in light of the benefit/harm ratio after taking into account the inaccuracies of PSA testing (and the associated morbidities of screen-recommended biopsies) and the overall low mortality from prostate cancer.

MRI is increasingly being used to characterize the prostate in patients with elevated PSA, with a negative predictive value (NPV) of 94% (for tumors with Gleason score of 7 or more), as described in W. C. Tsai, L. Field, S. Stewart, and M. Schultz, “Review of the accuracy of multi-parametric MRI prostate in detecting prostate cancer within a local reporting service,” J. Med. Imaging Radiat. Oncol., vol. 64, no. 3, pp, 379-384. Jun. 2020, doi: 10.1111/1754-9485.13029.

In comparison, community-based PSA screens achieve 78% NPV when the standard (4 ng/ml) threshold is used. MRI systems are increasingly being used to avoid unnecessary biopsy in patients with falsely-elevated PSAs, although the percentage of African-American men availing themselves of this modality is significantly less than for white men as described in E. L. Walton et al., “Barriers to obtaining prostate multi-parametric magnetic resonance imaging in African-American men on active surveillance for prostate cancer,” Cancer Med., vol. 8, no. 8, pp. 3659-3665, Jul. 2019, doi: 10.1002/cam4.2149. Avoiding biopsy may be particularly useful for African-American men, whose likelihood of major infections after prostate biopsy is higher than for white American men.

### SUMMARY

Disclosed embodiments provide an apparatus and method for pelvic imaging using a saddle shaped magnetic resonance imaging system.

In some embodiments, the apparatus is an array of electropermanent magnets having a longitudinal length that is longer than a width so that the subject may straddle the length of the array and sit on it. In some embodiments, a covering may be provided over the array for comfort while seated. In some embodiments, a method of collecting magnetic resonance images of a subject's pelvic region comprises providing an array of electropermanent magnets, positioning a subject in a straddle position over the array of electropermanent magnets; and imaging the subject's pelvic region.

### BRIEF DESCRIPTION OF FIGURES

Aspects and features of the invention are described in connection with various figures, in which:

FIG. 1 illustrates an array of at least two electropermanent magnets is configured in a saddle configuration, upon which a subject (thighs and buttock) sits to image a pelvic organ;

FIG. 2 is a block diagram of a control system and power source according to the disclosed embodiments; and

FIG. 3 is a flowchart of a method of imaging the pelvic region according to the disclosed embodiments.

### DETAILED DESCRIPTION

The present invention will now be described in connection with one or more embodiments. It is intended for the embodiments to be representative of the invention and not limiting of the scope of the invention. The invention is intended to encompass equivalents and variations, as should be appreciated by those skilled in the art.

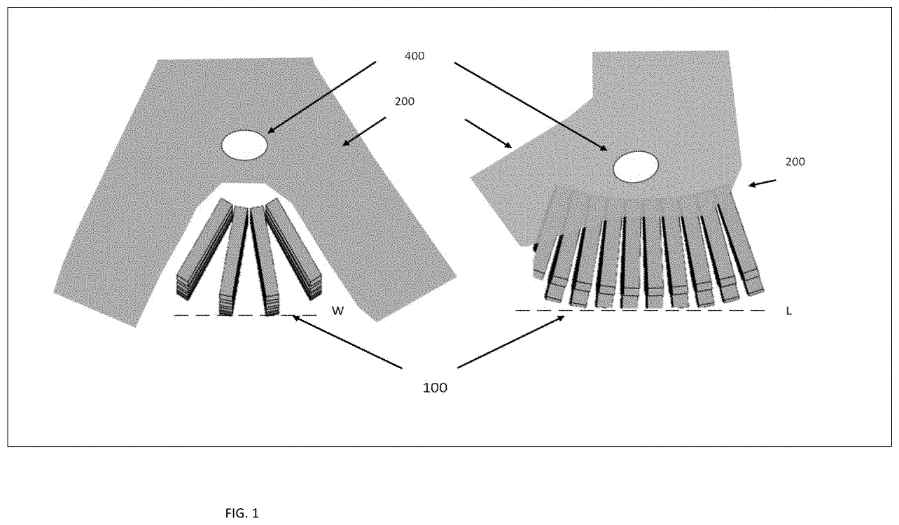

FIG. 1 illustrates an array 100 of at least two electropermanent magnets is configured in a saddle configuration, upon which a subject (thighs and buttock) sits to image a pelvic organ. As shown in FIG. 1, the illustrated apparatus is configured to perform a diagnostic MRI of a pelvic region, and in particular, pelvic organs, while a subject (either a human or non-human primate) is in a seated position. The array of electropermanent magnets 100 may be placed in a saddle configuration which is straddled by a subject whose thighs 200 are positioned, so that the pelvic organ of interest (for example, a prostate) 400 is in close proximity to the array. Front and side views of the subject 200 illustrate the subject seated on the array 100. The saddle configuration of electropermanent magnet array 100 has a longitudinal axis along with a subject straddles the array with more electropermanent magnets along its length L than the number of magnets along the width W, or cross section, of the array.

It is understood that a material may cover the arrays for purposes of comfort and safety. Likewise, it is understood that the array may be supported from underneath by a pedestal or other support, or from some other direction. In some embodiments Radio Frequency (RF) coils may be present to transmit electromagnetic energy into the pelvis and receive such energy from the pelvis, as described, for example, in U.S. Pat. No. 8,154,286, entitled “APPARATUS AND METHOD FOR DECREASING BIO-EFFECTS OF MAGNETIC FIELDS”, incorporated by reference in its entirety.

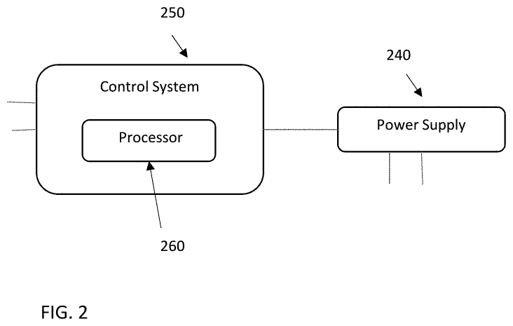

Alternatively, the electropermanent magnets in the array may be actuated in such sequence as to polarize and rotate spins so that a transmission RE coil is not needed for operation FIG. 2 is a block diagram of a control system and power source according to the disclosed embodiments. A power supply 240 and control system 250 including a processor 260 and switching and monitoring circuitry present to actuate the electropermanent magnets as seen in FIG. 2 and described in issued U.S. Pat. No. 10,908,240, entitled “METHOD FOR ACQUIRING AN IMAGE AND MANIPULATING OBJECTS WITH MAGNETIC GRADIENTS PRODUCED BY ONE OR MORE ELECTROPERMANENT MAGNET ARRAYS”, and in related patent applications including U.S. Pat. Pub. 20170227617, entitled “METHOD AND APPARATUS FOR MANIPULATING ELECTROPERMANENT MAGNETS FOR MAGNETIC RESONANCE IMAGING AND IMAGE GUIDED THERAPY”, incorporated herein by reference in their entirety. Images derived from the MRI may be analyzed with artificial intelligence.

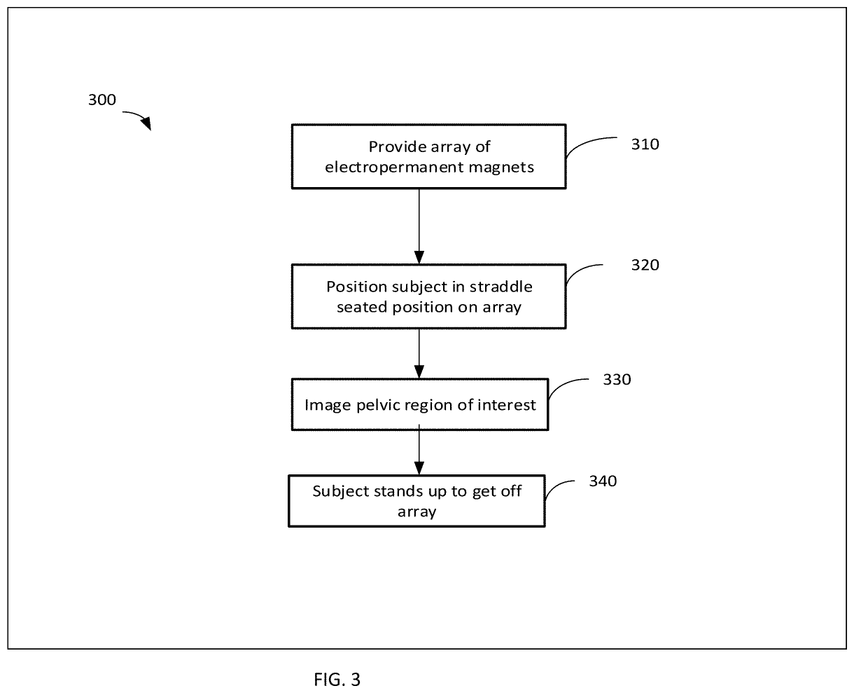

FIG. 3 is a flowchart of a method of imaging the pelvic region according to the disclosed embodiments. The method 300 of the disclosed embodiments may begin by providing an array of electropermanent magnets configured to be sat upon at 310. Subsequently, a subject may straddle and sits on the array of electropermanent magnets at 320. Thereafter, an MRI is collected by imaging the pelvic region of interest at 330. Following this, the subject gets off the array 340.

A barbershop model for health screening in the African-American population has been very successful in reducing hypertension, achieving 64% improvement as compared to controls. See, L. Margolis, “Inventing a New Model of Hypertension Care for Black Men,” N. Engl. J. Med., vol. 378, no. 14, pp. 1345-1347, Apr. 2018, doi: 10.1056/NEJMe1803106. This model is being applied to COVID testing in the Baltimore community by LifeBridge, a comprehensive health provider, in collaboration with Live Chair (an appointment booking cell phone application for barbershops).

Accordingly, booking (e.g., scheduling an appointment) and administration of the seated MRI procedure may be incorporated with another service such that it could be administered simultaneously or in conjunction with the service, e.g., while a subject waits for a haircut. In this manner, it is understood that the MRI saddle is configured to collect images from a subject while the subject is clothed.

Those skilled in the art will recognize, upon consideration of the above teachings, that the above exemplary embodiments and the control system may be based upon use of one or more programmed processors programmed with a suitable computer program. However, the disclosed embodiments could be implemented using hardware component equivalents such as special purpose hardware and/or dedicated processors. Similarly, general purpose computers, microprocessor based computers, micro-controllers, optical computers, analog computers, dedicated processors, application specific circuits and/or dedicated hard wired logic may be used to construct alternative equivalent embodiments.

Moreover, it should be understood that control and cooperation of the above-described components may be provided using software instructions that may be stored in a tangible, non-transitory storage device such as a non-transitory computer readable storage device storing instructions which, when executed on one or more programmed processors, carry out he above-described method operations and resulting functionality. In this case, the term “non-transitory” is intended to preclude transmitted signals and propagating waves, but not storage devices that are erasable or dependent upon power sources to retain information.

Those skilled in the art will appreciate, upon consideration of the above teachings, that the program operations and processes and associated data used to implement certain of the embodiments described above can be implemented using disc storage as well as other forms of storage devices including, hut not limited to non-transitory storage media (where non-transitory is intended only to preclude propagating signals and not signals which are transitory in that they are erased by removal of power or explicit acts of erasure) such as for example Read Only Memory (ROM) devices, Random Access Memory (RAM) devices, network memory devices, optical storage elements, magnetic storage elements, magneto-optical storage elements, flash memory, core memory and/or other equivalent volatile and non-volatile storage technologies without departing from certain embodiments. Such alternative storage devices should be considered equivalents.

While various exemplary embodiments have been described above, it should be understood that they have been presented by way of example only, and not limitation. Thus, the breadth and scope of the present invention should not be limited by any of the above-described exemplary embodiments, but should instead be defined only in accordance with the following claims and their equivalents.

1. An apparatus for collecting magnetic resonance images of a subject's pelvic region, the apparatus comprising:
an array of electropermanent magnets configured to be straddled by the subject during imaging, and to form an image of structures in the subject's pelvic region.

2. The apparatus of claim 1, wherein the array of electropermanent magnets are arranged in a saddle shape for the subject to be seated on during the imaging.

3. The apparatus of claim 2, wherein the array of electropermanent magnets has a longitudinal axis that is longer than a width of the array, wherein a cover is coupled to the array for the subject to sit on.

4. The apparatus of claim 1, wherein the apparatus further comprises power supply and switching and monitoring circuitry coupled to the electropermanent magnets.

5. A method of collecting magnetic resonance images of a subject's pelvic region, the method comprising:
providing an array of electropermanent magnets,
positioning a subject in a straddle position over the array of electropermanent magnets; and
imaging the subject's pelvic region

6. The method of claim 5, wherein the array of electropermanent magnets are arranged in a saddle-shape for the subject to he seated on during the imaging.

7. The apparatus of claim 6, wherein the array of electropermanent magnets has a longitudinal axis that is longer than a width of the array, wherein a cover is coupled to the array for the subject to sit on.

8. The method of claim 5, wherein the apparatus further comprises a power supply and switching and monitoring circuitry in order to perform magnetic resonance imaging.
